(19) United States
(12) Patent Application Publication
(10) Pub. No.: US 2003/0045541 A1
(43) Pub. Date: Mar. 6, 2003

(54) GABA-Receptor modulators with NMDA-Antagonistic activity

(76) Inventors: Christopher Bruckner, Berlin (DE); Rainer Dorow, Berlin (DE); Roland Neuhaus, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/198,045

(22) Filed: Jul. 19, 2002

Related U.S. Application Data
(60) Provisional application No. 60/313,019, filed on Aug. 20, 2001.

(30) Foreign Application Priority Data
Jul. 23, 2001 (DE) 10136842.9

(51) Int. Cl. A61K 31/4745

(52) U.S. Cl. 514/291

(57) ABSTRACT

The use of GABA_{A}-receptor modulators with NMDA-antagonistic activity for the production of a pharmaceutical agent for neuroprotection and the combination of GABA_{A}-receptor modulators with NMDA-antagonistic action and α-lipoic acid or dihydro-α-lipoic acid is described.

This application claims the benefit of the filing date of U.S. Provisional Application Serial No. 60/313,019 filed Aug. 20, 2001.

The invention relates to the use of GABA_{A}-receptor modulators with NMDA-antagonistic activity for the production of a pharmaceutical agent for neuroprotection and the combination of GABA_{A}-receptor modulators with NMDA-antagonistic action and α-lipoic acid or dihydro-α-lipoic acid.

Glutamate is an essential exciting transmitter in the human and mammalian body. Elevated glutamate levels lead to serious damage of the nerve tissue, which can lead to neurodegeneration. Within the glutamate receptors, the NMDA (N-methyl-D-aspartate) receptor plays the most important role pathologically. The NMDA receptor simultaneously acts in a stress- and ligand-dependent manner. At the normal resting potential of the neuron of −60 mV, the ion channel of the receptor is sealed by Mg²⁺ ions (Mayer, M. L. et al., Nature 1984; 309 (5965): 261-3), and a ligand cannot activate the receptor. If, however, the membrane is depolarized, Mg²⁺ leaves the ion channel, and the receptor can be activated by a ligand, which results in the inflow of Ca²⁺ and Na⁺, and the outflow of K⁺. The non-NMDA receptors are almost impermeable for Ca²⁺. An excessive activation of the NMDA receptor produces an intensified Ca²⁺ inflow, which has a cytotoxic effect. Apoptosis processes (Riveros, N. et al., Neuroscience 1986; 17 (3): 541-6) and later necrosis processes that ultimately lead to cell death are triggered by this Ca²⁺ inflow.

In the advanced stage of a neurodegenerative disease, the cells that deal with NMDA receptors preferably undergo necrosis, which results in that NMDA-receptor antagonists always lose more effectiveness with advancing neurodegeneration. The cells that carry the GABA receptors, however, are protected by the inhibitory action of this transmitter (GABA) and are also accessible to a pharmacological action in the state of advanced neurodegeneration.

Elevated intracellular Ca²⁺ levels can be caused by the above-described excessive activation of NMDA receptors. The latter frequently result in an enhanced imaging of free radicals, e.g., by the release of arachidonic acid or the conversion of xanthine-dehydrogenase to xanthine-oxidase. If elevated amounts of Ca²⁺ are taken up in the mitochondria, the latter can form hydroxyls and organic radicals (Packer, L. et al., Free Radio Biol Med 1997; 22 (1-2): 359-78). An enhanced activation of the NMDA receptor can lead to elevated intracellular nitrogen oxide levels, which can be reformed with superoxide to peroxynitrites and also are cytotoxic. A simultaneous use of a substance that has an NMDA-receptor-antagonistic effect with an antioxidant is suitable to prevent neurodegenerative damage in a synergistic way. It was possible to detect reduced glutathione levels in the cells of neurodegeneratively diseased mammals. Supplementing the glutathione would be desirable, but a significant resorption of glutathione from the nutrient or peroral input does not occur. A peroral input of α-lipoic acid, however, results in a considerable increase in the cellular glutathione level.

As neuroprotective therapy, the forms of treatment that result in a reduction or prevention of damage or death of neuronal cells are referenced. This can be carried out by reduction of the effects of an elevated glutamate level, e.g., by blocking the Ca²⁺ inflow into the cell, as well as by reduction of the release of glutamate.

The administration of NMDA-receptor antagonists would definitely be desirable for neuroprotective purposes, but is almost always accompanied in clinical tests by serious side effects, e.g., the so-called vacuolation of cells (Fritz, K. I. et al., Brain Res. 1999; 816 (2): 438-45), which have ultimately made a clinical introduction impossible. Moreover, almost all NMDA-receptor antagonists show moderately severe to severe psychiatric side effects. With simultaneous use of GABA-receptor-potentializing and NMDA-receptor-antagonizing active ingredients, a vacuolation cannot be observed (Olney, J. W. et al., Science 1991; 254 (5037): 1515-8).

GABA (gamma-amino-butyric acid) is a natural compound that is produced within the glutamate metabolism and represents the most important inhibitory transmitter of the mammal. A deficiency in GABA or a suppression of the GABA-ergic system results in most cases in spasms and epileptic seizures until cell death.

1,4-Benzodiazepines belong to the most widespread GABA_{A} receptor modulators. Compounds that consist of these substance groups, such as, e.g., midazolam and flunitrazepam, have a strong affinity to a special binding site for benzodiazepines, the benzodiazepine receptor. The latter is part of the GABA_{A} receptor. If GABA binds to the GABA_{A} receptor, this dissolves a chloride inflow. If benzodiazepines simultaneously bind to the benzodiazepine receptor, this chloride inflow is intensified. An intensified hyperpolarization of the cell results from this. As a mechanism for this purpose, an elevated opening probability of the chloride channel is expected. Binding of benzodiazepines to the benzodiazepine receptors intensifies the affinity of the GABA_{A} receptor to GABA and vice versa.

Benzodiazepines find a broad systemic use in the treatment of epilepsies, anxiety conditions, spasms and sleep disorders. Their toxic potential of danger is relatively low, since their effectiveness is limited by the amount of GABA that is present. Moreover, a more selective benzodiazepine-receptor antagonist, Flumazenil, exists, with which optional overdosages can be immediately antagonized. Also, barbituric acid derivatives, such as, e.g., phenobarbital, potentialize the opening of the GABA-chloride-ion channel.

It is known of β-carbolines that they have an affinity to the benzodiazepine receptors and exert an antagonistic, inversely agonistic and agonistic action based on the structure of the compounds in the properties known by the benzodiazepines. Many compounds from this substance class show only strong affinity to a specific binding site for benzodiazepines, which is part of the GABA_{A} receptor; many β-carbolines bind simultaneously to receptors for other neurotransmitters; and many β-carbolines bind only to receptors for other neurotransmitters and show no affinity to benzodiazepine receptors. It is thus described in WO 93/20820 that certain β-carbolines have an effect on the modulation site of the quisqualate receptor and correct the pathologically altered form of this receptor.

From an electrophysiological standpoint, an intensification of the GABA-ergic system results in a hyperpolarization of the cells. This makes more difficult a depolarization or the propagation of an action potential. Since the NMDA receptor is then only permeable for Ca²+ when the cell membrane is depolarized, an administration of GABA-intensified agents results in an inhibition of the NMDA receptor. By the hyperpolarization of the cell membrane, however, it prevents at the same time that stress-dependent Ca²⁺ channels are activated. These channels are not detected by pure NMDA-receptor antagonists.

GABA_{A}-receptor modulators change the affinity or the effectiveness of GABA on the receptor; in the absence of GABA, they are ineffective. The administration of GABA_{A}-receptor modulators is thus considerably less hazardous than that of agonists, since agonists have a stronger and stronger effect with increased administered concentration regardless of the amount of neurotransmitter that is present. The administration of GABA_{A}-receptor modulators, compared to GABA agonists, therefore has a considerably lower toxic risk.

The object was therefore to make available compounds that reduce or eliminate side effects that are known by NMDA antagonists and GABA agonists.

According to the invention, β-carbolines of formula I and physiologically compatible salt thereof

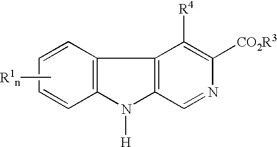

in which

R¹ is hydrogen or —O—R⁵,

R³ is hydrogen or C₁₋₄-alkyl,

R⁴ is hydrogen, C₁₋₄-alkyl or —CH₂—O—CH₃,

n is 1 or 2,

R⁵ is hydrogen, phenyl, benzyl, or phenyl that is substituted with Cl are suitable.

In each case, alkyl means straight-chain or branched alkyl such as methyl, ethyl, isopropyl, n-propyl, n-butyl, isobutyl, tert-butyl or sec-butyl.

R³ preferably stands for isopropyl. Substituent R¹ preferably stands in one place in 5- or 6-position or in two places in 6-, 7-position. Especially preferred embodiments are 5-(4-chlorophenoxy)-4-methoxymethyl-β-carboline-3-carboxylic acid isopropyl ester and especially 6-benzyloxy-4-methoxymethyl-β-carboline-3-carboxylic acid isopropyl ester (Abecarnil).

The production of the compounds of formula I and physiologically compatible salts thereof is carried out, for example, according to the processes described in EP 54507A, EP 239667A and EP 234173 or analogously to known methods.

GABA_{A}-receptor modulators that are suitable according to the invention and that have an NMDA-receptor-modulating action in addition to their GABA-receptor-modulating action are suitable for the neuroprotective therapy of neurodegenerative diseases, for example after stroke, cranio-cerebral trauma, and cerebral ischemia. In addition, those compounds are suitable for the therapy of other diseases of the central and peripheral nervous system, such as, e.g., Alzheimer's disease, Parkinson's disease, senile dementia, multiinfarct dementia; Huntington's disease, amyotrophic lateral sclerosis, restless leg syndrome, epilepsy, cell damages by hypoglycemia, hypoxia, and ischemia; neuronal damages, which are produced by uncontrolled movements; asphyxia as well as psychoses, schizophrenia, anxiety conditions, attacks of pain, migraines and vomiting; functional disorders such as impaired memory (amnesia), disturbances of the learning process, vigilance symptoms and deprivation symptoms after chronic intake of addictive agents such as benzodiazepines, hallucinogens, alcohol, cocaine or opiates; as well as multiple sclerosis; AIDS-induced encephalopathy and other infection-induced encephalopathies that are caused by rubella viruses, Herpes viruses, Borrelia and by unknown agents; Creutzfeldt-Jakob disease as well as neurodegenerative diseases of the peripheral nervous system such as polyneuropathies and polyneuritides.

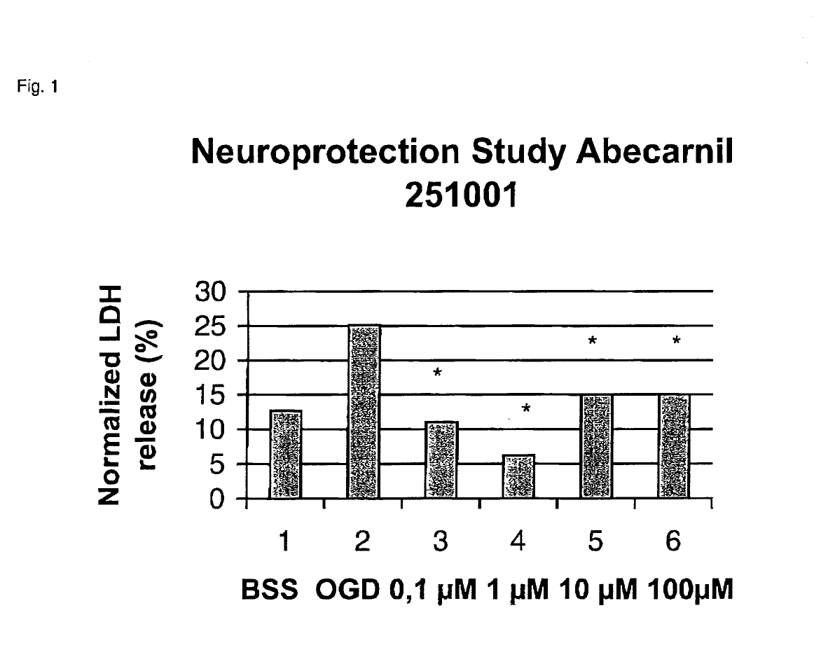

FIG. 1 shows the results that were obtained within the scope of the study of the neuroprotective properties of Abecarnil. The measurements were performed on primary cell cultures of cortical neurons of rats. After the cells were grown in culture, the following test was performed:

Glucose and oxygen were simultaneously removed from the culture solution. This model is a definitely sturdy model, which simulates the conditions during a very serious and very extensive stroke. In the zero comparison, the culture solution was left unchanged (BSS=balanced salt solution). In the control, oxygen and glucose were removed (OGD=oxygen and glucose deprivation); in the test group, Abecarnil was added simultaneously to the oxygen and glucose removal.

As damage parameters for the neurons, the LDH (lactate-dehydrogenase) level in the solution was measured by the cells. LDH is a definitely reliable stress parameter for the cells. The stronger the neuronal stress is or the more cells are already ruined, the more the LDH levels in the medium increase. From a pharmaceutical agent with a neuroprotective effect, it can be expected that it reduces the LDH level in the medium.

In FIG. 1, the values of such a measurement were depicted. If the LDH levels were below OGD 25.1, they were 11.0 with 0.1 μm of Abecarnil; 6.2 with 1 μm of Abecarnil; 14.9 with 10 μm of Abecarnil, and 15 with 100 μm of Abecarnil (after 24 hours). In the case of an Abecarnil concentration of 1 μm, this corresponds to an LDH reduction of over 75%!

Such a strong LDH reduction in the OGD model emphasizes the neuroprotective properties of Abecarnil and thus its suitability as agents for treating ischemic and neurodegenerative diseases such as, e.g., stroke.

β-Carbolines that are suitable according to the invention, such as Abecarnil, act via two receptor systems: on the one hand, they have a positive modulating effect on the benzodiazepine receptor and thus intensify the inhibitory action of GABA, but they simultaneously also have an NMDA-receptor-antagonistic effect, i.e., they reduce the harmful effects of glutamate. Both together produce a still more extensive neuroprotection than when using a pure GABA_{A}-receptor modulator.

The invention also relates to the use of the compounds of formula I for the production of a pharmaceutical agent for symptomatic and preventive treatment of the above-mentioned diseases of the central or peripheral nervous system.

The invention also comprises the combination of GABA_{A}-receptor modulators with NMDA-antagonistic action and α-lipoic acid or dihydro-α-lipoic acid. Together with its reduced form, dihydrolipoic acid (DHP), α-lipoic acid (1,2-dithiolane-3-valeric acid) forms a redox system. This redox system exerts a very strong antioxidative effect in the mammal organism. Moreover, α-lipoic acid binds free radicals, chelates metals and reactivates important cellular antioxidants and radical traps, such as, e.g., glutathione, vitamins C and E.

The combination treatment intensifies the neuroprotective effect and reduces the cytotoxic damage of an intensified Ca²+ inflow.

The pharmaceutical agents or compositions of the invention are produced with commonly used solid or liquid vehicles or diluents, and commonly used pharmaceutical and technical adjuvants corresponding to the desired type of administration with a suitable dosage in a way that is known in the art. Preferred preparations consist of a form for dispensing that is suitable for oral, enteral or parenteral administration, for example i.p. (intraperitoneal), i. v. (intravenous), i. m. (intramuscular) or percutaneous administration. Such forms for dispensing are, for example, tablets, film tablets, coated tablets, pills, capsules, powders, creams, ointments, lotions, liquids, such as syrups, gels, injectable liquids, for example for ip., i.v., i.m. or percutaneous injection, etc. In addition, depot forms, such as implantable preparations, as well as suppositories, are also suitable. In this case, the individual preparations deliver to the body the benzimidazole derivatives according to the invention in a gradual manner or the entire amount in a short time depending on their type.

For oral administration, capsules, pills, tablets, coated tablets and liquids or other known oral forms for dispensing can be used as pharmaceutical preparations. In this case, the pharmaceutical agents can be formulated in such a way that they release the active ingredients either in a short time and deliver them to the body, or they have a depot action, so that a longer-lasting, slow feed of active ingredient to the body is achieved. In addition to at least one benzimidazole derivative, the dosage units contain one or more pharmaceutically compatible vehicles, for example substances for adjusting the rheology of the pharmaceutical agent, surfactants, solubilizers, microcapsules, microparticles, granulates, diluents, binders, such as starch, sugar, sorbitol and gelatin, also fillers, such as silicic acid and talc, lubricant, dyes, perfumes and other substances.

Corresponding tablets can be obtained by, for example, mixing the active ingredient with known adjuvants, for example inert diluents such as dextrose, sugar, sorbitol, mannitol, polyvinylpyrrolidone, explosives such as corn starch or alginic acid, binders such as starch or gelatin, lubricants such as carboxypolymethylene, carboxymethyl cellulose, cellulose acetate phthalate or polyvinyl acetate. The tablets can also consist of several layers.

Accordingly, coated tablets can be produced by coating cores that are produced analogously to the tablets with agents that are commonly used in tablet coatings, for example polyvinyl-pyrrolidone or shellac, gum arabic, talc, titanium oxide or sugar. In this case, the coated tablet shell can also consist of several layers, whereby the adjuvants that are mentioned above in the case of the tablets can be used.

Capsules that contain active ingredients can be produced, for example, by the active ingredient being mixed with an inert vehicle such as lactose or sorbitol and encapsulated in gelatin capsules.

The active ingredients can also be formulated in the form of a solution, which is intended for oral administration and in addition to the active benzimidazole derivative contains as components a pharmaceutically compatible oil and/or a pharmaceutically compatible lipophilic surfactant and/or a pharmaceutically compatible hydrophilic surfactant and/or a pharmaceutically compatible water-miscible solvent.

To achieve a better bioavailability of the active ingredients according to the invention, the compounds can also be formulated as cyclodextrin clathrates. To this end, the compounds are reacted with α-, β- or γ-cyclodextrin or derivatives thereof.

If creams, ointments, lotions and liquids that can be applied externally are to be used, the latter must be constituted in such a way that the compounds according to the invention are fed to the body in a sufficient amount. In these forms for dispensing, adjuvants are contained, for example substances for adjusting the rheology of the pharmaceutical agents, surfactants, preservatives, solubilizers, diluents, substances for increasing the permeability for the benzimidazole derivatives according to the invention through the skin, dyes, perfumes and skin protection agents, such as conditioners and moisturizers. Together with the compounds according to the invention, other active ingredients can also be contained in the pharmaceutical agents (Ulmanns Enzyklopadie der technischen Chemie [Ullmanns' Encyclopedia of Technical Chemistry], Volume 4 (1953), pages 1-39; J. Pharm. Sci., 52, 918 ff. (1963); issued by Czetsch-Lindenwald, Hilfsstoffe für Pharmazie und angrenzende Gebiete [Adjuvants for Pharmaceutics and Related Fields]; Pharm. Ind., 2, 72 ff (1961); Dr. H. P. Fiedler, Lexikon der Hilfsstoffe für Pharmazie, Kosmetik und angrenzende Gebiete [Dictionary of Adjuvants for Pharmaceutics, Cosmetics and Related Fields], Cantor AG, Aulendorf/Württ., 1971].

The active ingredients can also be used in suitable solutions such as, for example, physiological common salt solution, as infusion or injection solutions. For parenteral administration, the active ingredients can be dissolved or suspended in a physiologically compatible diluent. As diluents, in particular oily solutions, such as, for example, solutions in sesame oil, castor oil and cottonseed oil, are suitable. To increase solubility, solubilizers, such as, for example, benzyl benzoate or benzyl alcohol, can be added.

To formulate an injectable preparation, any liquid vehicle can be used in which the compounds according to the invention are dissolved or emulsified. These liquids frequently also contain substances to regulate viscosity, surfactants, preservatives, solubilizers, diluents and other additives, with which the solution is set to isotonic.

It is also possible to incorporate the active ingredients in a transdermal system and thus to administer them transdermally. Such preparations can be formulated in such a way that a delayed release of active ingredients is made possible. To this end, known techniques can be used, for example depots that dissolve or that operate with a membrane. Implants can contain as inert materials, for example, biodegradable polymers or synthetic silicones, for example silicone gum.

The dosage of the active ingredients can vary depending on the type of application, the age and weight of the patient, the type and severity of the disease to be treated and similar factors. The daily dose can be given as a single dose to be administered once or divided into two or more daily doses. The compounds are introduced in a dosage unit of 0.05 to 100 mg of active substance in a physiologically compatible vehicle. In general, a dose of 0.1 to 500 mg/day, preferably 0.1 to 50 mg/day, is used.

In the combination preparations according to the invention, the active ingredients can be present in a formulation or else in respectively separate formulations, whereby the entire dose is administered once or divided into several doses.

The daily dose of the active ingredients in the combination preparations is 0.1 mg to 500 mg for the β-carboline derivative and 10 mg to 1000 mg for the α-lipoic acid or dihydro-α-lipoic acid; especially suitable are doses of 600 mg.

The effectiveness of the GABA_{A}-receptor modulators, which also have an NMDA-receptor-antagonizing effect at the same time, was determined by means of the tests described below:

The measurements were produced on cultivated neurons of Wistar embryos on the eighteenth day of gestation. After the preparation, the neurons were grown on small plates. To this end, small cover glasses for microscopic preparations with a diameter of 12 mm (assistant) were used. In each case four of the small cover glasses were transferred to a small plastic dish (Nuncion).

As preparation media, the following solutions were used: GBSS (Grey's buffered salt solution, Sigma) supplemented with 10 mmol of HEPES (N-2-hydroxyethyl-piperazine-N-2-ethanesulfonic acid, Sigma), adjusted to pH 7.3 with NaOH (Sigma). As culture media, the following were used: DMEM (Sigma), glucose-containing, equine serum 10% (Sigma) and glutamine. The cells were generally divided so that at the beginning of the culture, 50,000 cells per small plate were present. The medium was changed according to the preparation after 24 hours and then changed after three days in each case. Synaptic spontaneous activity developed generally starting from the tenth day in vitro.

The extracellular measuring solution had the following composition (in mmol): NaCl 140; KCl 5.4; CaCl₂ 2; HEPES 10; MgCl 1; glucose 25. The pH was set at 7.4 with NaOH. IPSCs were isolated by adding 1,2,3,4,-tetrahydro-6-nitro-2,3-di-oxo-benzo-quinoxaline-7-sulfonamide (NBQX 10 μm) and DL-2-amino-5-phosphonovaleric acid (±-APV 30 μm). EPSCs were isolated by adding bicuculline (10 μm) and picrotoxin (20 μm). In the examination of the EPSCs, a ringer solution without MgCl₂ was used. The pipette solution had the following composition (in mmol): KCl 120; MgCl₂ 2; CaCl₂ 1; HEPES 10; EGTA 11; glucose 20; the pH was 7.2.

The measurements were carried out at room temperature. As a reference, a silver-silver chloride pellet was used. −60 mV was selected as holding potential. Coming from the intensifier, the signals were reboosted with the aid of intermediate intensifiers and low-pass-filtered at 1 kHz. The thus modulated signals were visualized by means of a storage oscilloscope and recorded with a thermal plotter. At the same time, the signals were detected via a digital-to-analog converter and recorded on a videotape. The stored data were later played back, digitalized again and then evaluated “offline” with a PC. For evaluation, the TIDA program (HEKA, Germany) and the program set of J. Dempster (University of Strathclyde, UK) were used.

Inhibitory post-synaptic flows (IPSCs) were isolated by adding the glutamate-receptor antagonists NBQX (10 μm; for the AMPA receptor) and APV (30 μm; for the NMDA receptor). After adding the glutamate-receptor antagonists, the neuronal spontaneous activity collapsed almost completely. By adding 1 mmol of 4-amino-pyridine (4-AP), the activity could be stimulated again. Synaptic events were now shown that completely disappeared after bicuculline (20 μm) and picrotoxin (10 μm) were added and thus were purely GABA_{A}-receptor-mediated.

In addition, the pharmacological effects on the attenuation kinetics of the inhibitory post-synaptic flows (IPSCs) were examined here, since the latter is essential for the phenomenon of desensitization.

The amplitudes of the IPSCs fall off in a biexponential kinetics, i.e., a superposition of two components was found, whereby the first time constant represented the quickly decreasing portion of the IPSC and therefore is referred to as a τfast or fast time constant, while the second time constant describes the slower running portion of the IPSC and is therefore referred to as a τslow or slow time constant. The average attenuation time of the fast component τfast was 6.9±4.8 ms, and the slow component τslow was 34.1±12.5 ms. These values represent an average of all control measurements.

The effects of Abecarnil were very similar to those of a benzodiazepine, such as, e.g., midazolam (n=10). The frequency was thus reduced to 33.8±24.7% of the starting value (P<0.05); the amplitude was simultaneously increased (129.2=26.9%; P<0.05). The quick time constant of the averaged IPSCs showed a tendency toward increased values (143.9±51.0%), but these effects achieved no statistical significance. The slow time constant, however, was increased in a significant way, namely to 289.7±180.9% of the starting value (P<0.05).

Excitatory post-synaptic flows (EPSCs) were isolated by adding the GABA_{A}-receptor antagonists bicuculline (20 μm) and the GABA_{A}-receptor antagonist picrotoxin (10 μm) that has an allosteric effect. The addition of 4-AP also resulted here in an increase in neuronal activity. The pure EPSCs that can now be detected could be blocked completely by adding 10 μm of NBQX and 30 μm of APV and thus are purely glutamatergic.

The frequency of the EPSCs was reduced by Abecarnil to 32.6±19.8% of the starting value (P<0.05), and the amplitude was simultaneously reduced, namely to 81.2±17.8% of the starting value (P<0.05). In some cells, the frequency effect was only weakly pronounced, but in some cells the activity was blocked completely.

To study this first very surprising effect in more detail, the following test was performed: In addition, 30 μm of APV was applied on the isolated EPSCs to suppress the NMDA-controlled portion and to regard the AMPA-ergic activity as isolated. In this activity, Abecamil had no effect (n=3). If 10 μm of NBQX was applied counter to the isolated EPSCs to suppress the AMPA-ergic portion and to highlight the NMDA portion, and then 1 μm of Abecamil was applied, this activity was completely blocked by Abecarnil (n=7). It could thus be shown that Abecarnil has significant NMDA-antagonistic properties.

The preceding examples can be repeated with similar success by substituting the generically or specifically described reactants and/or operating conditions of this invention for those used in the preceding examples.

The entire disclosure of all applications, patents and publications, cited above and below, and of corresponding German Application No. 101 36 842.9, filed Jul. 23, 2001 is hereby incorporated by reference.

From the foregoing description, one skilled in the art can easily ascertain the essential characteristics of this invention and, without departing from the spirit and scope thereof, can make various changes and modifications of the invention to adapt it to various usages and conditions.

1. Use of GABA_{A}-receptor modulators with NMDA-antagonistic activity for the production of a pharmaceutical agent for neuroprotective treatment of neurodegenerative diseases of the central and peripheral nervous system.

2. Use according to claim 1 for neuroprotective treatment of diseases of the central nervous system.

3. Use according to claim 1 or 2 for treatment of multiple sclerosis, infection-induced encephalopathies or Creutzfeldt-Jakob disease.

4. Use of β-carboline derivatives of formula I and physiologically compatible salt thereof 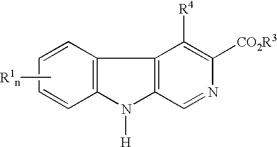
in which
R¹ is hydrogen or —O—R⁵,
R³ is hydrogen or C₁₋₄-alkyl,
R⁴ is hydrogen, C₁₋₄-alkyl or —CH₂—O—CH₃,
n is 1 or 2,
R⁵ is hydrogen, phenyl, benzyl, or phenyl that is substituted with Cl according to one of claims 1 to 3.

5. Use of 6-benzyloxy-4-methoxymethyl-β-carboline-3-carboxylic acid isopropyl ester according to one of claims 1 to 3.

6. Use of 6-benzyloxy-4-methoxymethyl-β-carboline-3-carboxylic acid isopropyl ester for the production of a pharmaceutical agent for neuroprotective treatment of neurodegenerative diseases selected from the group that comprises stroke, cerebral ischemia and cranio-cerebral trauma.

7. Active ingredient combination that comprises (1) a GABA_{A}-receptor modulator with NMDA-antagonistic activity and (2) α-lipoic acid or dihydro-α-lipoic acid.

8. Active ingredient combination according to claim 7 that consists of (1) 6-benzyloxy-4-methoxymethyl-β-carboline-3-carboxylic acid isopropyl ester and (2) α-lipoic acid or dihydro-α-lipoic acid.

9. Active ingredient combination according to claim 7 or 8 for neuroprotective treatment of neurodegenerative diseases of the central and peripheral nervous system.
